(19) United States
(12) Patent Application Publication
(10) Pub. No.: US 2026/0315378 A1
(43) Pub. Date: Oct. 15, 2026

(54) Oral Thin Film Formulations

(71) Applicant: Fitabeo Therapeutics Limited, Edinburgh (GB)

(72) Inventors: Issraa Rasheed AL-OBAIDI, Edinburgh (GB); Alexander Balfour MULLEN, Edinburgh (GB)

(21) Appl. No.: 19/483,314

(22) PCT Filed: May 9, 2024
(86) PCT No.: PCT/GB2024/051206

§ 371 Date: Nov. 11, 2025

(30) Foreign Application Priority Data
May 11, 2023 (GB) 2306966.9

(51) Int. Cl. A61K 9/00; A61K 45/06; A61K 47/10; A61K 47/12; A61K 47/32; A61K 47/38

(52) U.S. Cl. CPC A61K 9/006; A61K 45/06; A61K 47/10; A61K 47/12; A61K 47/32; A61K 47/38

(57) ABSTRACT

There are disclosed compositions which are suitable for preparing an orally dissolvable film which has improved properties, and in particular one or more of good elasticity (low brittleness), fast disintegration, and low stickiness. Also disclosed are orally dissolvable films made from such compositions, and medical uses and methods of treatment using such orally dissolvable films.

### FIELD OF THE INVENTION

The present invention relates to a drug delivery system, and in particular, to a drug delivery system in the form of an oral thin film.

### BACKGROUND

Many medical and/or pathological conditions require administration of chemical and/or pharmaceutical substances or drugs to a subject. An example of a common application requiring the delivery of a drug is the treatment of pain.

A known method of administering a substance orally to a patient is by the use of an Oral Thin Film (‘OTF’). The term “Oral Thin Film” is a term known in the art, and the term “thin” will not be understood as a relative term, but merely as part of the recognised phrase “Oral Thin Film” which refers to a type of product widely understood by a person of ordinary skill in the art as having a recognised meaning. Other synonymous terms to describe the same type of product are “Oral Dissolvable Films”, “Thin Dissolving Films”, “Orodispersible Films”, “Orally Consumable Films”, “Oral Drug Strips” or “Oral Films”.

OTFs are films capable of dissolving rapidly in the mouth of a subject, and which are principally composed of water soluble film forming polymer(s). OTFs were first developed as breath fresheners and have progressed towards the oral delivery of active pharmaceutical ingredients (APIs). Approximately the size of a postage stamp, they dissolve quickly in saliva when placed in the mouth of a subject without the need to chew or drink water. These films are formulated using minimal food or pharmaceutical grade excipients and are inexpensive to produce. As an alternative solid platform for drug delivery, they can be formulated without the need for preservatives or solvents; an attractive option for paediatric drug delivery where the administration of liquid formulations can result in exposure to potentially harmful excipients.

Typically, 5-30% drug loading can be achieved with oral thin films and the target drug content can be easily controlled by adjusting the film dimensions (R.P. Dixit, S.P. Puthli, Oral strip technology: overview and future potential, J Control Release, 139 (2009) 94-107). Generally, the target surface area of the oral thin film is in the range of 5-20 cm² to allow for practical dose administration (A. Arya, A. Chandra, V. Sharma, K. Pathak, Fast dissolving oral films: an innovative drug delivery system and dosage form, Int J ChemTech Res, 2 (2010) 576-583). Film thickness can also be easily controlled. Numerous different film-forming polymers are available with different disintegration and release rates or even mucoadhesive properties. Many taste masking technologies as well as sweeteners and flavourings can be included to produce an acceptable, palatable drug-delivery system.

Typical OTF formulations include:
a film-forming polymer (binder) such as xanthan gum, pullulan or HPMC;
a disintegrating agent such as polyvinyl pyrrolidone (PVP);
an emulsifier such a sucrose ester, e.g. Sisterna™;
a thickener such as starch or chitosan;
a plasticizer such as glycerol;
water.

Typical OTF formulations may comprise other excipients including sweeteners, flavorants, etc.

However, there are a number of problems associated with conventional OTF formulations. These include in particular:
brittleness;
stickiness upon introduction in a subject's mouth (particularly problematic for infants or geriatric patients); and
slow disintegration.

Examples of OTF formulations which suffer from one or more of these problems are disclosed in CN 102160858 A, CN 107823191 A, CN 113712940 A, CN 110755412 A, US2015038594 A1, CN 103720676 A, CN 108785280 A, US 2015/328125 A1, WO 2022/152883 A1, US 2012/009260 A1, US 2015/125495 A1, US 2007/0281003 A1, and US 2019/380973 A1.

It is an object of the invention to address and/or mitigate one or more problems associated with the prior art.

It is an object of the invention to provide an oral thin film having improved properties.

### SUMMARY

The present invention is based on the finding that it is possible to provide an OTF which has improved properties, and in particular having one or more of the following properties: good elasticity (low brittleness), fast disintegration, and low stickiness, and preferably exhibiting all of these properties. It was found that this can be achieved by the non-obvious selection of the following:
selecting a film-forming polymer having high viscosity (but not so high as to prevent casting of the OTF);
removing thickeners and emulsifiers found in conventional formulations as these are believed to adversely affect disintegration but are typically considered a requirement in the art; and/or
increasing the proportion of disintegrant and/or plasticizer in the formulation to mitigate the absence of thickeners and emulsifiers.

According to a first aspect, there is provided a composition suitable for an oral thin film, wherein the composition comprises:
(i) polyvinyl alcohol (PVA);
(ii) glycerol; and
(iii) polyethylene glycol (PEG),
wherein the combined amount of glycerol and polyethylene glycol is about 12 to 24 wt % in the dry formulation and the glycerol and polyethylene glycol are in a weight ratio of about 1:2.1 to 1:4. In particular embodiments, the PEG has an average molecular weight ranging from about 1100 to about 2900 g·mol⁻¹ or about 1,300 to about 2,000 g·mol⁻¹.

Thus, in the composition, the polyvinyl alcohol may constitute or may act as a film-forming agent. In an embodiment, the polyvinyl alcohol may constitute or may act as the film-forming agent, i.e. no further film-forming agent may be present.

The glycerol and polyethylene glycol may constitute or may act as plasticizers.

The inventors has surprisingly found that the formulation comprising a combination of polyvinyl alcohol (PVA) as a film-forming agent, and glycerol and polyethylene glycol as plasticizers, wherein the combined amount of glycerol and polyethylene glycol is about 12 to 24 wt % in the dry formulation and the glycerol and polyethylene glycol are in a weight ratio of about 1:2.1 to 1:4, provides an oral thin film which has good elasticity (low brittleness), fast disintegration, and low stickiness.

The inventors have found that the physical properties of the film (e.g. the elasticity, fast disintegration, and low stickiness) are particularly favourable where the PEG has an average molecular weight ranging from about 1100 to about 2900 g·mol⁻¹, more specifically about 1,300 to about 2,000 g·mol⁻¹, such as about 1,500 g·mol⁻¹. Further, it was surprisingly found that the present composition does not require the inclusion of thickeners and emulsifiers in order to achieve those properties in the OTF.

Typically, the composition may comprise about 30 to about 50 wt % dry weight of polyvinyl alcohol. Typically, the composition may comprise about 10 to about 18 wt % wet weight of polyvinyl alcohol.

The term “dry weight” will be herein understood as an amount or ratio of an ingredient based on the total weight of the composition without the solvent. The term “wet weight” will be herein understood as an amount or ratio of an ingredient based on the total weight of the composition including the solvent.

Typically, the composition may comprise about 3 to about 17 wt % dry weight of glycerol. Typically, the composition may comprise about 1 to about 6 wt % wet weight of glycerol.

Typically, the composition may comprise about 3 to about 17 wt % dry weight of polyethylene glycol. Typically, the composition may comprise about 1.1 to about 6.3 wt % wet weight of polyethylene glycol.

Typically, the polyvinyl alcohol may have a viscosity, e.g. kinematic viscosity, of about 3.0 to about 3.8 mm²/s at 20° C., in a 4% aqueous solution. Advantageously, this provides the composition with viscosity sufficiently high to avoid the need to use a thickener, but not so high as to prevent casting of the composition into an OTF.

In some embodiments, the combined amount of glycerol and polyethylene glycol is about 12 to about 20 wt % in the dry formulation or about 4 to about 8 wt % in the wet formulation. In some embodiments, the combined amount of glycerol and polyethylene glycol is about 13 to about 18 wt % in the dry formulation or about 4.5 to about 7.5 wt % in the wet formulation. In some embodiments, the combined amount of glycerol and polyethylene glycol is about 14 to about 17 wt % in the dry formulation or about 5 to about 7 wt % in the wet formulation.

In particular embodiments, the glycerol and polyethylene glycol are in a weight ratio of about 1:2.1 to 1:3.5, about 1:2.4 to 1:3.2, or about 1:2.7 to 1:2.9.

The polyvinyl alcohol may have a degree of hydrolysis of about 86.5 to about 89.0 mol %.

In some embodiments, the polyethylene glycol has an average molecular weight, e.g. number molecular weight of about 1100 to about 2900 dalton or g·mol⁻¹, such as about 1100 to about 2700 dalton or g·mol⁻¹, about 1100 to about 2500 dalton or g·mol⁻¹ or about 1100 to about 2300 dalton or g·mol⁻¹. The polyethylene glycol may have an average molecular weight, e.g. number molecular weight, ranging from 1,000 to 2,000, e.g. from 1,300 to 1,600, dalton or g·mol⁻¹. In some embodiments, the polyethylene glycol has an average molecular weight, e.g. number molecular weight, ranging from about 1,300 to about 2,000 dalton or g·mol⁻¹, about 1,300 to about 1,700 dalton or g·mol⁻¹, or about 1500 dalton or g·mol⁻¹.

The composition may further comprise hydroxypropyl methylcellulose (HPMC). In such instance, the HPMC may constitute or may act as a film-forming agent. Thus, the composition may comprise two or more forming agents including at least PVA and HPMC. In an embodiment, the composition may comprise two forming agents including at least PVA and HPMC, and no further film-forming agent may be present.

The composition may comprise about 10 to about 15 wt % dry weight of HPMC. The composition may comprise about 3.1 to about 4.7 wt % wet weight of HPMC.

In some embodiments, the combined amount of polyvinyl alcohol and hydroxypropyl methylcellulose is about 40 to about 65 wt % in the dry formulation, or about 13 to about 21 wt % in the wet formulation. In some embodiments, the combined amount of polyvinyl alcohol and hydroxypropyl methylcellulose is about 45 to about 60 wt % in the dry formulation, or about 15 to about 19 wt % in the wet formulation. For example, the combined amount of polyvinyl alcohol and hydroxypropyl methylcellulose 15 may be about 50 to about 55 wt % in the dry formulation, or about 16 to about 18 wt % in the wet formulation.

According to a second aspect, there is provided a composition suitable for an oral thin film, wherein the composition comprises:
(i) hydroxypropyl methylcellulose (HPMC);
(ii) polyvinyl pyrrolidone (PVP);
(iii) glycerol; and
(iv) polyethylene glycol (PEG).

Thus, in the composition, the HPMC may constitute or may act as a film-forming agent. In an embodiment, the HPMC may constitute or may act as the film-forming agent, i.e. no further film-forming agent may be present.

The polyvinyl pyrrolidone (PVP) may constitute or may act as a disintegration agent. In an embodiment, the PVP may constitute or may act as the disintegration agent, i.e. no further disintegration agent may be present.

The glycerol and polyethylene glycol may constitute or may act as plasticizers.

The inventors has surprisingly found that the formulation comprising a combination of HPMC as a film-forming agent, PVP as a disintegration agent, and glycerol and polyethylene glycol as plasticizers, provides an oral thin film which has good elasticity (low brittleness), fast disintegration, and low stickiness.

Further, it was surprisingly found that the present composition does not require the inclusion of thickeners and emulsifiers in order to achieve those properties in the OTF.

Typically, the hydroxypropyl methylcellulose may comprise about 19 to about 24 wt % methoxy groups and about 7 to about 12 wt % hydroxypropyl groups.

Typically, the hydroxypropyl methylcellulose may have a dynamic viscosity of about 3500 to about 5600 cPs at 20° C. in a 2% aqueous solution.

In an embodiment, the hydroxypropyl methylcellulose may comprise, may consist essentially of or may consist of METHOCEL™ K4M.

The composition may comprise about 60 to about 66 wt % dry weight of hydroxypropyl methylcellulose. The composition may comprise about 4.1 to about 4.5 wt % wet weight of hydroxypropyl methylcellulose.

The composition may comprise about 4 to about 8 wt % dry weight of polyvinyl pyrrolidone. The composition may comprise about 0.27 to about 0.54 wt % wet weight of polyvinyl pyrrolidone.

The composition may comprise about 4 to about 20 wt % dry weight of glycerol. The composition may comprise about 0.2 to about 1.4 wt % wet weight of glycerol.

The composition may comprise about 4 to about 20 wt % dry weight of polyethylene glycol. The composition may comprise about 0.2 to about 1.4 wt % wet weight of polyethylene glycol.

In some embodiments, the polyethylene glycol has an average molecular weight, e.g. number molecular weight of about 1100 to about 2900 dalton or g·mol⁻¹, such as about 1100 to about 2700 dalton or g·mol⁻¹, about 1100 to about 2500 dalton or g·mol⁻¹ or about 1100 to about 2300 dalton or g·mol⁻¹. The polyethylene glycol may have an average molecular weight, e.g. number molecular weight, ranging from 1,000 to 2,000, e.g. from 1,300 to 1,600, dalton or g·mol⁻¹. In some embodiments, the polyethylene glycol has an average molecular weight, e.g. number molecular weight, ranging from about 1,300 to about 2,000 dalton or g·mol⁻¹, about 1,300 to about 1,700 dalton or g·mol⁻¹, or about 1500 dalton or g·mol⁻¹.

The composition may comprise glycerol and polyethylene glycol in a combined amount of about 12 to about 24 wt % in the dry formulation. The composition may comprise glycerol and polyethylene glycol in a combined amount of about 0.5 to about 1.5 wt % in the wet formulation.

The composition may comprise polyethylene glycol and glycerol in a weight ratio of about 1:1.5 to about 1:4, such as 1:2.1 to about 1:4.

The composition may comprise polyvinyl pyrrolidone having a dynamic viscosity of about 5.5 to about 8.5 cPs at 20° C. in a 10% aqueous solution. The PVP may be commercially known as “K30”. The PVP may be defined as a water-soluble homopolymer of N-vinyl-2-pyrrolidone.

According to a third aspect, there is provided a composition suitable for an oral thin film, wherein the composition comprises:
(i) hydroxypropyl methylcellulose (HPMC) comprising about 19 to about 24 wt % methoxy groups and about 7 to about 12 wt % hydroxypropyl groups;
(ii) polyvinyl pyrrolidone (PVP); and
(iii) glycerol; and/or polyethylene glycol (PEG).

Thus, in the composition, the HPMC may constitute or may act as a film-forming agent. In an embodiment, the HPMC may constitute or may act as the film-forming agent, i.e. no further film-forming agent may be present.

The polyvinyl pyrrolidone (PVP) may constitute or may act as a disintegration agent. In an embodiment, the PVP may constitute or may act as the disintegration agent, i.e. no further disintegration agent may be present.

The glycerol and/or polyethylene glycol may constitute or may act as plasticizer(s).

The inventors have surprisingly found that the formulation comprising a combination of HPMC as a film-forming agent, PVP as a disintegration agent, and glycerol and/or polyethylene glycol as plasticizer(s), provides an oral thin film which has good elasticity (low brittleness), fast disintegration, and low stickiness. Further, it was surprisingly found that the present composition does not require the inclusion of thickeners and emulsifiers in order to achieve those properties in the OTF.

The composition may comprise glycerol and polyethylene glycol.

Typically, the hydroxypropyl methylcellulose may have a dynamic viscosity of about 3500 to about 5600 cPs at 20° C. in a 2% aqueous solution.

In an embodiment, the hydroxypropyl methylcellulose may comprise, may consist essentially of or may consist of METHOCEL™ K4M.

The composition may comprise about 60 to about 66 wt % dry weight of hydroxypropyl methylcellulose. The composition may comprise about 4.1 to about 4.5 wt % wet weight of hydroxypropyl methylcellulose.

The composition may comprise about 4 to about 8 wt % dry weight of polyvinyl pyrrolidone. The composition may comprise about 0.27 to about 0.54 wt % wet weight of polyvinyl pyrrolidone.

The composition may comprise about 4 to about 20 wt % dry weight of glycerol. The composition may comprise about 0.2 to about 1.4 wt % wet weight of glycerol.

The composition may comprise about 4 to about 20 wt % dry weight of polyethylene glycol. The composition may comprise about 0.2 to about 1.4 wt % wet weight of polyethylene glycol.

In some embodiments, the polyethylene glycol has an average molecular weight, e.g. number molecular weight of about 1100 to about 2900 dalton or g·mol⁻¹, such as about 1100 to about 2700 dalton or g·mol⁻¹, about 1100 to about 2500 dalton or g·mol⁻¹ or about 1100 to about 2300 dalton or g·mol⁻¹. The polyethylene glycol may have an average molecular weight, e.g. number molecular weight, ranging from 1,000 to 2,000, e.g. from 1,300 to 1,600, dalton or g·mol⁻¹. In some embodiments, the polyethylene glycol has an average molecular weight, e.g. number molecular weight, ranging from about 1,300 to about 2,000 dalton or g·mol⁻¹, about 1,300 to about 1,700 dalton or g·mol⁻¹, or about 1500 dalton or g·mol⁻¹.

The composition may comprise glycerol and polyethylene glycol in a combined amount of about 12 to about 24 wt % in the dry formulation. The composition may comprise glycerol and polyethylene glycol in a combined amount of about 0.5 to about 1.5 wt % in the wet formulation.

The composition may comprise polyethylene glycol and glycerol in a weight ratio of about 1:1.5 to about 1:4, such as 1:2.1 to about 1:4.

The composition may comprise polyvinyl pyrrolidone having a dynamic viscosity of about 5.5 to about 8.5 cPs at 20° C. in a 10% aqueous solution. The PVP may be commercially known as “K30”. The PVP may be defined as a water-soluble homopolymer of N-vinyl-2-pyrrolidone.

According to a fourth aspect, there is provided a composition suitable for an oral thin film, wherein the composition comprises:
(i) hydroxypropyl methylcellulose (HPMC);
(ii) polyvinyl pyrrolidone (PVP); and
(iii) glycerol; and/or polyethylene glycol (PEG),
wherein the composition comprises about 60 to about 66 wt % hydroxypropyl methylcellulose in the dry formulation and/or about 4 to about 8 wt % polyvinyl pyrrolidone in the dry formulation,
and wherein the composition does not comprise an emulsifier.

Thus, in the composition, the HPMC may constitute or may act as a film-forming agent. In an embodiment, the HPMC may constitute or may act as the film-forming agent, i.e. no further film-forming agent may be present.

The polyvinyl pyrrolidone (PVP) may constitute or may act as a disintegration agent. In an embodiment, the PVP may constitute or may act as the disintegration agent, i.e. no further disintegration agent may be present.

The glycerol and/or polyethylene glycol may constitute or may act as plasticizer(s).

The inventors has surprisingly found that the formulation comprising a combination of HPMC as a film-forming agent, PVP as a disintegration agent, and glycerol and/or polyethylene glycol as plasticizers, wherein the composition comprises about 60 to about 66 wt % hydroxypropyl methylcellulose in the dry formulation and/or about 4 to about 8 wt % polyvinyl pyrrolidone in the dry formulation, and wherein the composition does not comprise an emulsifier, provides an oral thin film which has good elasticity (low brittleness), fast disintegration, and low stickiness.

Further, it was surprisingly found that the present composition does not require the inclusion of thickeners and emulsifiers in order to achieve those properties in the OTF.

The composition may comprise about 4.1 to about 4.5 wt % wet weight of hydroxypropyl methylcellulose.

The composition may comprise about 0.27 to about 0.54 wt % wet weight of polyvinyl pyrrolidone.

The composition may comprise glycerol and polyethylene glycol.

Typically, the hydroxypropyl methylcellulose may comprise about 19 to about 24 wt % methoxy groups and about 7 to about 12 wt % hydroxypropyl groups.

Typically, the hydroxypropyl methylcellulose may have a dynamic viscosity of about 3500 to about 5600 cPs at 20° C. in a 2% aqueous solution.

In an embodiment, the hydroxypropyl methylcellulose may comprise, may consist essentially of or may consist of METHOCEL™ K4M.

The composition may comprise about 4 to about 20 wt % dry weight of glycerol. The composition may comprise about 0.2 to about 1.4 wt % wet weight of glycerol.

The composition may comprise about 4 to about 20 wt % dry weight of polyethylene glycol. The composition may comprise about 0.2 to about 1.4 wt % wet weight of polyethylene glycol.

In some embodiments, the polyethylene glycol has an average molecular weight, e.g. number molecular weight of about 1100 to about 2900 dalton or g·mol⁻¹, such as about 1100 to about 2700 dalton or g·mol⁻¹, about 1100 to about 2500 dalton or g·mol⁻¹ or about 1100 to about 2300 dalton or g·mol⁻¹. The polyethylene glycol may have an average molecular weight, e.g. number molecular weight, ranging from 1,000 to 2,000, e.g. from 1,300 to 1,600, dalton or g·mol⁻¹. In some embodiments, the polyethylene glycol has an average molecular weight, e.g. number molecular weight, ranging from about 1,300 to about 2,000 dalton or g·mol⁻¹, about 1,300 to about 1,700 dalton or g·mol⁻¹, or about 1500 dalton or g·mol⁻¹.

The composition may comprise glycerol and/or polyethylene glycol in a combined amount of about 12 to about 24 wt % in the dry formulation. The composition may comprise glycerol and/or polyethylene glycol in a combined amount of about 0.5 to about 1.5 wt % in the wet formulation.

The composition may comprise glycerol and polyethylene glycol in a combined amount of about 12 to about 24 wt % in the dry formulation. The composition may comprise glycerol and polyethylene glycol in a combined amount of about 0.5 to about 1.5 wt % in the wet formulation.

The composition may comprise polyethylene glycol and glycerol in a weight ratio of about 1:1.5 to about 1:4, such as 1:2.1 to about 1:4.

The composition may comprise polyvinyl pyrrolidone having a dynamic viscosity of about 5.5 to about 8.5 cPs at 20° C. in a 10% aqueous solution. The PVP may be commercially known as “K30”. The PVP may be defined as a water-soluble homopolymer of N-vinyl-2-pyrrolidone.

The following may apply to any previous aspects:
Preferably, the composition may not comprise a thickener. Typical thickeners may include starch, gelatin, chitosan, agar, carrageenan, xanthan gum, guar gum, pectin, alginates, etc

Preferably, the composition may not comprise an emulsifier. Typical emulsifiers may include polysorbates such as polysorbate 80 (also known as polyoxyethylene (20) sorbitan monooleate, available commercially e.g., as Tween® 80), mono- and diglycerides, sucrose ester, lecithins, etc.

The composition may further comprise optional additives such as one or more of a filler, flavouring agent, sweetener, colorant, antioxidant, microbial preservatives, and the like.

The composition may comprise a pharmaceutically acceptable solvent.

The composition may comprise about 50 to about 98 wt %, e.g. from about 60 to about 95 wt % solvent.

The solvent may comprise, may consist essentially of or may consist of water. The solvent may comprise or may further include an organic solvent, e.g. ethanol. In some embodiments, the solvent may comprise, may consist essentially of or may consist of water and ethanol.

The composition may comprise a salivary stimulant. The salivary stimulant may comprise, may consist essentially of or may consist of citric acid.

The composition may comprise from 0 to about 20 wt %, e.g. from about 1 to about 20 wt %, e.g. from about 2 to about 18 wt % dry weight of salivary stimulant. The composition may comprise from 0 to about 7.5 wt %, e.g. from about 0.1 to about 7.5 wt %, e.g. from about 0.2 to about 7 wt % wet weight of salivary stimulant.

The composition may comprise a sweetener. The sweetener may comprise, may consist essentially of or may consist of a natural sweetener such as sucrose, or an artificial sweetener such as sucralose, saccharin, aspartame or isomalt. In some embodiments, the sweetener may act as a salivary stimulant.

The composition may comprise about 0 to about 20 wt %, e.g. from about 1 to about 20 wt %, e.g. from about 2 to about 15 wt % dry weight of sweetener. The composition may comprise about 0 to about 8 wt %, e.g. from about 0.1 to about 8 wt %, e.g. from about 0.2 to about 5 wt % wet weight of sweetener. The composition may comprise a flavouring agent. The flavouring agent may comprise, may consist essentially of or may consist of a natural or artificial flavouring agent such as spearmint oil or lemon flavour. Any suitable flavouring agent may be used, such as orange, passion fruit, tutti fruitti, strawberry, lime, raspberry, or cherry flavourant.

The composition may comprise about 0 to about 20 wt %, e.g. from about 1 to about 15 wt %, e.g. from about 2 to about 12 wt % dry weight of flavouring agent. The composition may comprise about 0 to about 8 wt %, e.g. from about 0.1 to about 8 wt %, e.g. from about 0.2 to about 5 wt % wet weight of flavouring agent.

The composition may further comprise a pharmaceutically active ingredient. The pharmaceutically active ingredient may comprise or may be a therapeutic agent. In other words, the term “pharmaceutically active ingredient” may be herein understood as referring to an active ingredient capable of having a therapeutic effect on a patient, that is, understood to be capable of treating a disease and/or a medical condition.

In an embodiment, the pharmaceutically active ingredient may comprise or may consist of a drug or substance capable of alleviating pain in a subject, e.g. an analgesic.

The pharmaceutically active ingredient may comprise or may consist of an opioid drug and/or derivative thereof. The pharmaceutically active ingredient may comprise or may consist of any other drug class. The pharmaceutically active ingredient may comprise or may consist of one or more of alfentanil, allylprodine, alphaprodine, amfepramone, amphetamine, amphetaminil, anileridine, apocodeine, apomorphine, asimadoline, axomadol, benzylmorphine, bezitramide, bremazocine, brifentanil, buprenorphine, butorphanol, carfentanil, clonitazene, codeine, cyclazocine, cyclorphan, cyprenorphine, cyprodime, deltorphin, desomorpine, dextromethorphan, dextromoramide, dextropropoxyphene, dezocine, diamorphine, diamorphone, diampromide, diacetylmorphine, dihydrocodeine, dihydrocodeinone, dihydroetophine, dihydromorphine, dimenoxadol, dimephetamol, dimepheptanol, dimethylthiambutene, dioxyaphetyl butyrate, diphenoxylate, dipipanone, diprenorphine, dronabinol, Met-enkephalin, Leu-enkephalin, dynorphin A, dynorphin B, β-endorphin, eptazocine, eridorphin, ethoheptazine, 14-ethoxymetopon, ethylketocyclazocine, ethylmethylthiambutene, ethylmorphine, etonitazene, etorphine, extromoramide, fencamfamine, fenethylline, fenproporex, fentanyl, a-methylfentanyl, beta-funaltrexamine, P-hydroxy-3-methylfentanyl, heroin, hydrocodone, hydromorphone, hydromorphodone, hydroxymethylmorphinan, hydroxypethidine, isomethadone, ketobemidone, kyotorphin, levo-a-acetylmethadol, levacetylmethadol, levallorphan, levomethadone, levomethadyl acetate, levallorphan, levorphanol, levophenacylmorphan, lofentanil, Lofexidine, loperamide, malbuphine, mazindol, melatonin, mefenorex, meperidine, meprobamate, meptazinol, metazocine, methadone, metopon, methyldihydromorphine, methyldihydromorphinone, methyhnorphine, methylnaltrexone, methylphenidate (and stereoisomers), methyprylon, metopon, mirfentanil, modafmil, morphiceptin, morphinan, morphine, myrophine, nabilone, nalbuphine, nalbuphine, nalmefene, nalorphine, naloxone, naloxone benzoylhydrazone, naltrexone, naltriben, naltrindole, naltrindole isothiocyanate, narceine, natbuphine, nicomorphine, nor-binaltorphimine, norlevorphanol, normethadone, nalorphine, normorphine, norpipanone, noscapine, ohmefentanyl, o-methylnaltrexone, opium, onitazene, oxycodone, oxymorphone, papaveretum, papaverine, paregoric, pemoline, pemoline, pentazocine, pethidine, phendimetrazine, phendimetrazone, phenmetrazine, phenadoxone, phenomorphan, phenazocine, phenoperidine, pholcodeine, piminodine, pipradrol, piritramide, prenorphine, profadol, properidine, propheptazine, promedol, properidine, propiram, propoxyphene, propylhexedrine, remifentanil, spiradoline, sufentanil, sufentanyl, tapentadol, thebaine, tramadol, trefentanil, tilidine, viminol or their salts, or opioids of the phenanthrene, morphinan, benzomorphan, methadone, phenylpiperidine, propionanilide 4-anilidopiperidine, 4-aryl piperidines, 4-heteroarylpiperidines class, and the like, and/or salts and derivatives thereof.

In an embodiment, the pharmaceutically active ingredient may be morphine, or a salt or derivative thereof. In another embodiment, the pharmaceutically active ingredient may be apomorphine.

The pharmaceutically active ingredient may comprise, may consist essentially of, or may consist of a drug or substance capable of treating and/or preventing of metabolic bone disease in infants, e.g. hypophosphataemia, a condition common to low birth weight infants and associated with osteopenia of prematurity. The pharmaceutically active ingredient may comprise, may consist essentially of, or may consist of a potassium salt, e.g., potassium acid phosphate.

The pharmaceutically active ingredient may comprise, may consist essentially of, or may consist of a drug or substance capable of treating and/or preventing stomach and/or oesophageal pathological condition, e.g., vomiting, nausea, reflux, or the like. The pharmaceutically active ingredient may comprise, may consist essentially of, or may consist of a metoclopramide and/or derivative thereof, e.g., metoclopramide hydrochloride. The pharmaceutically active ingredient may comprise, may consist essentially of, or may consist of cyclizine, ondansetron and/or granisetron, which may typically be used to treat nausea.

The pharmaceutically active ingredient may comprise, may consist essentially of, or may consist of a drug or substance capable of treating and/or preventing erectile dysfunction or progressive diseases of the nervous system which can affect locomotion such as Parkinson's disease.

The pharmaceutically active ingredient may comprise, may consist essentially of, or may consist of cannabis or a cannabis derivative such a cannabidiol and/or other cannabinoids.

The pharmaceutically active ingredient may comprise, may consist essentially of, or may consist of melatonin.

The formulations described herein may find application in the treatment of humans, especially paediatrics and/or geriatrics, for example, where swallowing of a pill may be problematic. Another area is in the veterinary field where administering pills to animals can be difficult to achieve.

The composition comprises from about 0.01 to about 10 wt %, e.g. from about 0.1 to about 1 wt %, dry weight of the pharmaceutically active ingredient or from about 1×10⁻⁵ to about 0.01 wt %, e.g. from about 1×10⁻⁴ to about 0.001 wt %, wet weight of the pharmaceutically active ingredient.

In an embodiment of the first aspect, there is provided a composition suitable for an oral thin film, wherein the composition comprises:
(i) polyvinyl alcohol (PVA) in an amount of about 30 to about 50 wt % dry weight of the composition;
(ii) glycerol in an amount of about 3 to about 17 wt % dry weight of the composition; and
(iii) polyethylene glycol (PEG) in an amount of about 3 to about 17 wt % dry weight of the composition,
wherein the combined amount of glycerol and polyethylene glycol is about 12 to 24 wt % in the dry formulation and the glycerol and polyethylene glycol are in a weight ratio of about 1:2.1 to 1:4. Typically, the polyethylene glycol has an average molecular weight ranging from about 1100 to about 2900 g·mol⁻¹ or about 1,300 to about 2,000 g·mol⁻¹.

The composition may comprise about 10 to about 18 wt % wet weight of polyvinyl alcohol.

The composition may comprise about 1 to about 6 wt % wet weight of glycerol.

The composition may comprise about 1.1 to about 6.3 wt % dry weight of polyethylene glycol.

The composition may comprise water in an amount of about 50 to about 80 wt %, e.g. about 60 to about 70 wt % of the total (wet) composition.

The composition may comprise from about 10 to about 18 wt % dry weight of salivary stimulant. The composition may comprise from about 3 to about 7 wt % wet weight of salivary stimulant.

The composition may comprise from about 5 to about 15 wt %, e.g. about 10 to about 15 wt % dry weight of sweetener. The composition may comprise from about 1 to about 6 wt %, e.g. about 4 to about 6 wt % wet weight of sweetener.

The composition may comprise from about 5 to about 15 wt %, e.g. from about 10 to about 15 wt % dry weight of flavouring agent. The composition may comprise from about 1 to about 6 wt %, e.g. about 4 to about 6 wt % wet weight of flavouring agent.

In an embodiment of the second aspect, there is provided a composition suitable for an oral thin film, wherein the composition comprises:
(i) hydroxypropyl methylcellulose (HPMC) in an amount of about 60 to about 66 wt % dry weight of the composition;
(ii) polyvinyl pyrrolidone (PVP) in an amount of about 4 to about 8 wt % dry weight of the composition;
(iii) glycerol in an amount of about 4 to about 20 wt % dry weight of the composition; and
(iv) polyethylene glycol (PEG) in an amount of about 4 to about 20 wt % dry weight of the composition.

The composition may comprise water in an amount of about 70 to about 95 wt %, e.g. about 80 to about 95 wt % of the total (wet) composition.

The composition may comprise from about 1 to about 10 wt % dry weight of salivary stimulant. The composition may comprise from about 0.1 to about 1 wt % wet weight of salivary stimulant.

The composition may comprise from about 2 to about 10 wt % dry weight of sweetener. The composition may comprise from about 0.1 to about 7 wt % wet weight of sweetener.

The composition may comprise from about 1 to about 12 wt % dry weight of flavouring agent. The composition may comprise from about 0.1 to about 1 wt % wet weight of flavouring agent.

In an embodiment of the third aspect, there is provided a composition suitable for an oral thin film, wherein the composition comprises:
(i) hydroxypropyl methylcellulose (HPMC) comprising about 19 to about 24 wt % methoxy groups and about 7 to about 12 wt % hydroxypropyl groups, in an amount of about 60 to about 66 wt % dry weight of the composition;
(ii) polyvinyl pyrrolidone (PVP) in an amount of about 4 to about 8 wt % dry weight of the composition; and
(iii) glycerol; and/or polyethylene glycol (PEG) each in an amount of about 4 to about 20 wt % dry weight of the composition.

The composition may comprise water in an amount of about 70 to about 95 wt %, e.g. about 80 to about 95 wt % of the total (wet) composition.

The composition may comprise from about 1 to about 10 wt % dry weight of salivary stimulant. The composition may comprise from about 0.1 to about 1 wt % wet weight of salivary stimulant.

The composition may comprise from about 2 to about 10 wt % dry weight of sweetener. The composition may comprise from about 0.1 to about 7 wt % wet weight of sweetener.

The composition may comprise from about 1 to about 12 wt % dry weight of flavouring agent. The composition may comprise from about 0.1 to about 1 wt % wet weight of flavouring agent.

In an embodiment of the fourth aspect, there is provided a composition suitable for an oral thin film, wherein the composition comprises:
(i) hydroxypropyl methylcellulose (HPMC) in an amount of about 60 to about 66 wt % dry weight of the composition;
(ii) polyvinyl pyrrolidone (PVP) in an amount of about 4 to about 8 wt % dry weight of the composition; and
(iii) glycerol; and/or polyethylene glycol (PEG) each in an amount of about 4 to about 20 wt % dry weight of the composition,
wherein the composition comprises about 60 to about 66 wt % hydroxypropyl methylcellulose in the dry formulation and/or about 4 to about 8 wt % polyvinyl pyrrolidone in the dry formulation,
and wherein the composition does not comprise an emulsifier.

The composition may comprise water in an amount of about 70 to about 95 wt %, e.g. about 80 to about 95 wt % of the total (wet) composition.

The composition may comprise from about 1 to about 10 wt % dry weight of salivary stimulant. The composition may comprise from about 0.1 to about 1 wt % wet weight of salivary stimulant.

The composition may comprise from about 2 to about 10 wt % dry weight of sweetener. The composition may comprise from about 0.1 to about 7 wt % wet weight of sweetener.

The composition may comprise from about 1 to about 12 wt % dry weight of flavouring agent. The composition may comprise from about 0.1 to about 1 wt % wet weight of flavouring agent.

According to a fifth aspect, there is provided an orally dissolvable film comprising or made from a composition according to any of the preceding aspects.

According to a sixth aspect, there is provided a composition according to any of the first to fourth aspects or an orally dissolvable film according to the fifth aspect, for use as a medicament.

According to a seventh aspect, there is provided a composition according to any of the first to fourth aspects or an orally dissolvable film according to the fifth aspect, for use in the treatment of one or more conditions or diseases selected from pain, migraine, headache, inflammation, metabolic bone disease, hypophosphataemia, nausea, vomiting, gastroesophageal reflux disease, anxiety, dyspnoea, sleep disorders, erectile dysfunction, anxiety, and/or movement disorders e.g. Parkinson's.

The composition or OTF may be for use in treating pain.

According to an eighth aspect, there is provided a method of treating one or more conditions or diseases selected from pain, migraine, headache, inflammation, metabolic bone disease, hypophosphataemia, nausea, vomiting, gastroesophageal reflux disease, anxiety, dyspnoea, sleep disorders, erectile dysfunction, anxiety and/or movement disorders e.g. Parkinson's, the method comprising administering to a patient in need thereof an orally dissolvable film according to the fifth aspect.

The method may comprise treating pain.

According to a ninth aspect, there is provided a method of preparing an orally dissolvable film, the method comprising casting on a support a composition according to any of the first to fourth aspects.

The method may comprise or may be a method of preparing an orally dissolvable film according to the fifth aspect.

The method may comprise evaporating at least part of the solvent.

It will be appreciated that the final orally dissolvable film may include some residual solvent, e.g. water. Therefore, the method may comprise evaporating some of the solvent present in the composition. It will be appreciated that the amount of residual solvent, e.g. water, in the OTF may depend on the type and amount of the components used in the composition.

The features described in relation to any aspect of the invention may equally apply to any other aspect and, merely for brevity, are not repeated. For example, features described in relation to compositions can apply in relation to methods, and vice versa.

The invention may be further described with reference to the following non-limiting clauses:
1. A composition suitable for an oral thin film, wherein the composition comprises:
(i) polyvinyl alcohol;
(ii) glycerol; and
(iii) polyethylene glycol,
wherein the combined amount of glycerol and polyethylene glycol is about 12 to 24 wt % in the dry formulation and the glycerol and polyethylene glycol are in a weight ratio of about 1:2.1 to 1:4.
2. The composition of clause 1, wherein the polyethylene glycol has an average molecular weight ranging from about 1100 to about 2900 dalton or g·mol⁻¹, about 1100 to about 2700 dalton or g·mol⁻¹, about 1100 to about 2500 dalton or g·mol⁻¹, about 1100 to about 2300 dalton or g·mol⁻¹, about 1,300 to about 2,000 g·mol⁻¹, about 1,300 to about 1,700 g·mol⁻¹, or about 1500 g·mol⁻¹.
3. The composition of clause 1 or clause 2, wherein the combined amount of glycerol and polyethylene glycol is about 12 to about 20 wt %, about 13 to about 18 wt % or about 14 to about 17 wt % in the dry formulation.
4. The composition of any one of clauses 1 to 3, wherein the glycerol and polyethylene glycol are in a weight ratio of about 1:2.1 to 1:3.5, about 1:2.4 to 1:3.2, or about 1:2.7 to 1:2.9.
5. The composition of any one of clauses 1 to 4, wherein the composition does not comprise a thickener.
6. The composition of any one of clauses 1 to 5, wherein the composition does not comprise an emulsifier.
7. The composition of any one preceding clause, wherein the composition comprises about 30 to about 50 wt % dry weight of polyvinyl alcohol or from about 10 to about 18 wt % wet weight of polyvinyl alcohol.
8. The composition of any one preceding clause, wherein the composition comprises about 3 to about 7 wt % dry weight of glycerol or from about 1 to about 6 wt % wet weight of glycerol.
9. The composition of any one preceding clause, wherein the composition comprises about 3 to about 17 wt % dry weight of polyethylene glycol or from about 1.1 to about 6.3 wt % dry weight of polyethylene glycol.
10. The composition of any one preceding clause, wherein the composition comprises a pharmaceutically acceptable solvent.
11. The composition of clause 10, wherein the solvent is water.
12. The composition of clause 10 or clause 11, wherein the composition comprises about 60 to about 70 wt % solvent of the total (wet) composition.
13. The composition of any one preceding clause, further comprising a salivary stimulant.
14. The composition of clause 13, wherein the composition comprises about 10 to about 18 wt % salivary stimulant or from about 3 to about 7 wt % wet weight of salivary stimulant.
15. The composition of any one preceding clause, further comprising a sweetener.
16. The composition of clause 15, wherein the composition comprises about 10 to about 15 wt % dry weight of sweetener or from about 1 to about 6 wt %, e.g. about 4 to about 6 wt % wet weight of sweetener.
17. The composition of any one preceding clause, further comprising a flavouring agent.
18. The composition of clause 17, wherein the composition comprises about 10 to about 15 wt % dry weight of flavouring agent or from about 4 to about 6 wt % wet weight of flavouring agent.
19. The composition of any one preceding clause, further comprising hydroxypropyl methylcellulose.
20. The composition of clause 19, wherein the composition comprises about 10 to about 15 wt % dry weight of hydroxypropyl methylcellulose or from about 3.1 to about 4.7 wt % wet weight of HPMC.
21. The composition of clause 19 or clause 20, wherein the combined amount of polyvinyl alcohol and hydroxypropyl methylcellulose is about 40 to about 65 wt %, about to about 60 wt % or about 50 to about 55 wt % in the dry formulation.
22. The composition of any one preceding clause, wherein the polyvinyl alcohol has a kinematic viscosity of about 3.0 to about 3.8 mm²/s at 20° C., in a 4% aqueous solution.
23. A composition suitable for an oral thin film, wherein the composition comprises:
(i) hydroxypropyl methylcellulose;
(ii) polyvinyl pyrrolidone;
(iii) glycerol; and
(iv) polyethylene glycol.
24. The composition of clause 23, wherein the hydroxypropyl methylcellulose comprises about 19 to about 24 wt % methoxy groups and about 7 to about 12 wt % hydroxypropyl groups.
25. The composition of clause 23 or clause 24, wherein the hydroxypropyl methylcellulose has a dynamic viscosity of about 3500 to about 5600 cPs at 20° C. in a 2% aqueous solution.
26 A composition suitable for an oral thin film, wherein the composition comprises:
(i) hydroxypropyl methylcellulose comprising about 19 to about 24 wt % methoxy groups and about 7 to about 12 wt % hydroxypropyl groups;
(ii) polyvinyl pyrrolidone; and
(iii) glycerol; and/or polyethylene glycol.
27 The composition of clause 26, wherein the hydroxypropyl methylcellulose has a dynamic viscosity of about 3500 to about 5600 cPs at 20° C. in a 2% aqueous solution.
28. The composition of any one of clauses 23 to 27, wherein the composition comprises about 60 to about 66 wt % dry weight of hydroxypropyl methylcellulose, or about 4.1 to about 4.5 wt % wet weight of hydroxypropyl methylcellulose.
29. The composition of any one of clauses 23 to 28, wherein the composition comprises about 4 to about 8 wt % dry weight of polyvinyl pyrrolidone or about 0.27 to about 0.54 wt % wet weight of polyvinyl pyrrolidone.
30. The composition of any one of clauses 23 to 29, wherein the composition does not comprise an emulsifier.
31 A composition suitable for an oral thin film, wherein the composition comprises:
(i) hydroxypropyl methylcellulose;
(ii) polyvinyl pyrrolidone; and
(iii) glycerol; and/or polyethylene glycol, wherein the composition comprises about 60 to about 66 wt % hydroxypropyl methylcellulose in the dry formulation and/or about 4 to about 8 wt % polyvinyl pyrrolidone in the dry formulation, and wherein the composition does not comprise an emulsifier.
32. The composition of clause 31, wherein the composition comprises glycerol and polyethylene glycol.
33. The composition of clause 31 or clause 32, wherein the hydroxypropyl methylcellulose comprises about 19 to about 24 wt % methoxy groups and about 7 to about 12 wt % hydroxypropyl groups.
34. The composition of any one of clauses 31 to 33, wherein the hydroxypropyl methylcellulose has a dynamic viscosity of about 3500 to about 5600 cPs at 20° C. in a 2% aqueous solution.
35. The composition of any one of clauses 23 to 34, wherein the composition comprises about 4 to about 20% dry weight of glycerol or about 0.2 to about 1.4 wt % wet weight of glycerol.
36. The composition of any one of clauses 23 to 35, wherein the composition comprises about 4 to about 20% dry weight of polyethylene glycol or about 0.2 to about 1.4 wt % wet weight of polyethylene glycol.
37. The composition of any one of clauses 23 to 36, wherein the composition comprises polyethylene glycol which has an average molecular weight ranging from 1,300 to 1,600 gmol-1.
38. The composition of any one of clauses 23 to 37, wherein the composition comprises glycerol and polyethylene glycol in a combined amount of about 12 to about 24 wt % in the dry formulation or of 0.5 to about 1.5 wt % in the wet formulation.
39. The composition of any one of clauses 23 to 38, wherein the composition comprises polyethylene glycol and glycerol in a weight ratio of about 1:1.5 to about 1:4, such as 1:2.1 to about 1:4.
40. The composition of any one of clauses 23 to 39, wherein the composition comprises polyvinyl pyrrolidone which has a dynamic viscosity of about 5.5 to about 8.5 cPs at 20° C. in a 10% aqueous solution.
41. The composition of any one of clauses 23 to 40, wherein the composition comprises a pharmaceutically acceptable solvent.
42. The composition of clause 41, wherein the solvent is water.
43. The composition of clause 41 or clause 42, wherein the composition comprises about 80 to about 95 wt % solvent.
44. The composition of any one of clauses 23 to 43, further comprising a sweetener.
45. The composition of clause 44, wherein the composition comprises about 2 to about 10 wt % dry weight of sweetener or from about 0.1 to about 7 wt % wet weight of sweetener.
46. The composition of any one of clauses 23 to 45, further comprising a flavouring agent.
47. The composition of clause 46, wherein the composition comprises about 1 to about 12 wt % dry weight of flavouring agent or from about 0.1 to about 1 wt % wet weight of flavouring agent.
48. The composition of any one of clauses 23 to 47, further comprising a salivary stimulant.
49. The composition of clause 48, wherein the composition comprises about 1 to about 10 wt % dry weight salivary stimulant, or from about 0.1 to about 1 wt % wet weight of salivary stimulant.
50. The composition of any one preceding clause, further comprising a pharmaceutically active ingredient.
51. The composition of clause 50, wherein the composition comprises from about 0.01 to about 10 wt %, e.g. from about 0.1 to about 1 wt %, dry weight of the pharmaceutically active ingredient, or from about 1×10⁻⁵ to about 0.01 wt %, e.g. from about 1×10⁻⁴ to about 0.001 wt %, wet weight of the pharmaceutically active ingredient.
52. The composition of clause 50 or clause 51, wherein the pharmaceutically active ingredient is an opioid.
53. An orally dissolvable film comprising or made from a composition according to any preceding clause.
54. A composition as defined in any one of clauses 50 to 52 or an orally dissolvable film according to claim 53 for use as a medicament.
55. A composition as defined in any one of clauses 50 to 52 or an orally dissolvable film according to claim 53 for use in the treatment or prophylaxis of pain.
56. A method of treating pain comprising administering to a patient in need thereof an orally dissolvable film according to clause 53.
57. A method of preparing an orally dissolvable film, the method comprising casting on a support a composition according to any of clauses 1 to 52.

### DETAILED DESCRIPTION

In the present disclosure, reference is made to a number of terms, which have the meanings provided below, unless a context indicates to the contrary. The nomenclature used herein for defining compounds, in particular the compounds according to the invention, is in general based on the rules of the IUPAC organisation for chemical compounds, specifically the “IUPAC Compendium of Chemical Terminology (Gold Book)”. For the avoidance of doubt, if a rule of the IUPAC organisation is in conflict with a definition provided herein, the definition herein is to prevail. Furthermore, if a compound structure is in conflict with the name provided for the structure, the structure is to prevail.

The term “comprising” or variants thereof is to be understood herein to imply the inclusion of a stated element, integer or step, or group of elements, integers or steps, but not the exclusion of any other element, integer or step, or group of elements, integers or steps.

The term “consisting” or variants thereof is to be understood to imply the inclusion of a stated element, integer or step, or group of elements, integers or steps, and the exclusion of any other element, integer or step or group of elements, integers or steps.

The term “about” herein, when qualifying a number or value, is used to refer to values that lie within +5% of the value specified. For example, if a temperature is specified to be about 5 to about 13° C., temperatures of 4.75 to 13.65° C. are included.

Reference to physical states of matter (such as liquid or solid) refer to the matter's state at 25° C. and atmospheric pressure unless the context dictates otherwise.

As explained above, the present inventors have discovered that it is possible to provide an orally dissolvable film which has improved properties, and in particular having one or more of the following properties: good elasticity (low brittleness), fast disintegration, and low stickiness, and preferably exhibiting all of these properties. It was found that this can be achieved by the non-obvious selection of the following:
selecting a film-forming polymer having high viscosity (but not so high as to prevent casting of the OTF);
removing thickeners and emulsifiers found in conventional formulations as these are believed to adversely affect disintegration but are typically considered a requirement in the art; and/or
increasing the proportion of disintegrant and/or plasticizer in the formulation to mitigate the absence of thickeners and emulsifiers.

Non-limiting examples of formulations found to achieve such results are described below.

### EXAMPLES

### Example 1

TABLE 1
Formulation of Composition “F64”

| | | Percentage Dry | | Percentage |
|---|---|---|---|---|
| | | weight (Without | | Wet weight |
| Excipients | Used as | Drug) % w/w | | (%) |
| Hydroxypropyl | Water Soluble | 66 | (60-66%) | 4.48 |
| Methylcellulose | Polymer | | | |
| (Methocel ™MK4M | | | | |
| Premium) | | | | |
| Sucralose powder | Sweetener | 3.8 | | 0.255 |
| Polyvinyl | Disintegrant | 5.9 | (4-8%) | 0.399 |
| Pyrrolidine | | | | |
| (PVP K30) | | | | |
| Glycerol | Plasticiser | 11 | (4-20%) | 0.74 |
| Polyethylene | Plasticiser | 4.6 | (4-20%) | 0.31 |

| | | | |
|---|---|---|---|
| Glycol | | | |
| (PEG1500) | | | |
| Spearmint oil | Flavouring | 7.7 | 0.56 |
| flavour | agent | | |

| | | |
|---|---|---|
| Distilled Water | Amount of water used was | 93.26 |
| | 12.6 gm from total weight | |
| | with excipients 13.51 g | |

TABLE 2
Formulation of Composition “F73”

| | | Percentage Dry | | Percentage |
|---|---|---|---|---|
| | | weight (Without | | Wet weight |
| Excipients | Used as | Drug) % w/w | | (%) |
| Hydroxypropyl | Water soluble | 65 | (60-66%) | 4.48 |
| Methylcellulose | polymer | | | |
| (Methocel ™K4M | | | | |
| Premium) | | | | |
| Isomalt | Sweetener | 5.4 | | 0.368 |
| Polyvinyl | Disintegrant | 5.9 | (4-8%) | 0.399 |
| Pyrrolidine | | | | |
| (PVP K30) | | | | |
| Glycerol | Plasticiser | 10.8 | (4-20%) | 0.739 |
| Glycol | | | | |
| Polyethylene | Plasticiser | 4.5 | (4-20%) | 0.31 |

| | | | |
|---|---|---|---|
| Glycol | | | |
| (PEG1500) | | | |
| Spearmint | Flavouring | 7.5 | 0.569 |
| | agent | | |

| | | |
|---|---|---|
| Distilled | Amount of water used was | 93.19 |
| Water | 12.6 gm from total weight | |
| | with excipients 13.52 g | |

TABLE 3
Formulation of Composition “F74”

| | | Percentage Dry | | Percentage |
|---|---|---|---|---|
| | | weight (Without | | Wet weight |
| excipients | Used as | Drug) % w/w | | (%) |
| Hydroxypropyl | Water soluble | 62 | (60-66%) | 4.46 |
| Methylcellulose | polymer | | | |
| (Methocel ™MK4M | | | | |
| Premium) | | | | |
| Isomalt | Sweetener | 5.1 | | 0.36 |
| Polyvinyl | Disintegrant | 5.59 | (4-8%) | 0.397 |
| Pyrrolidine | | | | |
| (PVP K30) | | | | |
| Glycerol | Plasticiser | 10 | (4-20%) | 0.736 |
| Polyethylene | Plasticiser | 4.6 | (4-20%) | 0.309 |

| | | | |
|---|---|---|---|
| Glycol | | | |
| (PEG1500) | | | |
| Spearmint | Flavouring | 7.2 | 0.567 |
| | agent | | |
| Citric acid | Salivary | 4.4 | 0.316 |
| | stimulant | | |

| | | |
|---|---|---|
| Distilled | Amount of water used was | 92.85 |
| Water | 12.6 gm from total weight | |
| | with excipients 13.57 g | |

The Formulations of Tables 1, 2 and 3 were based on Hydroxypropyl Methylcellulose (and in particular Methocel™ K4M) as film-former. It was found that this type of film-forming polymer had a viscosity which allowed the formation of an OTF with superior properties without the need for thickeners and emulsifiers.

The Formulations of Tables 1, 2 and 3 also included PVP as a disintegration agent, and further included glycerol and/or polyethylene glycol as plasticizers. These formulations were found to provide an oral thin film which has good elasticity (low brittleness), fast disintegration, and low stickiness.

Further, it was surprisingly found that the present composition does not require the inclusion of thickeners and emulsifiers in order to achieve those properties in the OTF.

TABLE 4
Formulation of Composition “F71”

| | | Percentage Dry | | Percentage |
|---|---|---|---|---|
| | | weight (Without | | Wet weight |
| excipients | Used as | Drug) % w/w | | (%) |
| PVA 03P (Gohsenol ™ | Water | 43 | (30-50%) | 15.89 |
| EG-03P from Mitsubishi | soluble | | | |
| Chemical Corporation) | polymer | | | |
| Sucralose | Sweetener | 12 | | 4.45 |
| Glycerol | Plasticiser | 4.3 | (3-17%) | 1.58 |
| Polyethylene | Plasticiser | 12 | (3-17%) | 4.45 |
| Glycol | | | | |
| (PEG1500) | | | | |
| Spearmint | Flavouring | 12 | | 4.45 |
| | agent | | | |
| Citric acid | Salivary | 15 | | 5.56 |
| | Stimulant | | | |

| | | |
|---|---|---|
| Distilled | Amount of water used was | 63.59 |
| Water | 4 gm from total weight with | |
| | excipients 6.29 g | |

TABLE 5
Formulation of Composition “F75”

| | | Percentage Dry | | Percentage |
|---|---|---|---|---|
| | | weight (Without | | Wet weight |
| Excipients | Used as | Drug) % w/w | | (%) |
| PVA 03P (Gohsenol ™ | Water | 39 | (30-50%) | 13.31 |
| EG-03P from Mitsubishi | soluble | | | |
| Chemical Corporation) | polymer | | | |
| Hydroxypropyl | Water | 13 | (10-15%) | 4.06 |
| Methylcellulose | soluble | | | |
| (Methocel ™K4M | polymer | | | |
| Premium) | | | | |
| Sucralose | Sweetener | 11 | | 3.44 |
| Glycerol | Plasticiser | 3.9 | (3-17%) | 1.23 |
| Polyethylene | Plasticiser | 11 | (3-17%) | 3.44 |
| Glycol | | | | |
| (PEG1500) | | | | |

| | | | |
|---|---|---|---|
| Spearmint | Flavouring | 11 | 3.44 |
| | agent | | |
| Citric acid | Salivary | 13.8 | 4.31 |
| | stimulant | | |

| | | |
|---|---|---|
| Distilled | Amount of water used was | 67.73 |
| Water | 5.5 gm from total weight | |
| | with excipients 8.12 g | |

The Formulations of Tables 4 and 5 were based on polyvinyl alcohol (PVA) as film-former. It was found that this type of film-forming polymer had a viscosity which allowed the formation of an OTF with superior properties without the need for thickeners and emulsifiers.

The Formulations of Tables 4 and 5 also included glycerol and polyethylene glycol as plasticizers. These formulations were found to provide an oral thin film which has good elasticity (low brittleness), fast disintegration, and low stickiness.

It was surprisingly found that the Formulations of Tables 4 and 5 did not require the use of PVP as a disintegration agent.

Further, it was surprisingly found that the present composition does not require the inclusion of thickeners and emulsifiers in order to achieve those properties in the OTF.

Whilst the Formulation of Table 4 used polyvinyl alcohol (PVA) as the sole film-forming polymer, the Formulation of Table 5 used a mixture of PVA and HPMC.

TABLE 6
Formulation of Composition “F64” including an API

| | | | | Percentage |
|---|---|---|---|---|
| | | | | weight |
| | | | Percentage | (% w/w) |
| | | Weight | weight | of the |
| Excipients | Used as | (g) | (% w/v) | cast film |
| Hydroxypropyl | Water | 0.606 | 4.48 | 66 |
| Methylcellulose | Soluble | | | |
| (Methocel ™MK4M | Polymer | | | |
| Premium) | | | | |
| Sucralose powder | Sweetener | 0.0345 | 0.255 | 3.8 |
| Polyvinyl | Disinte- | 0.054 | 0.399 | 5.9 |
| Pyrrolidine | grant | | | |
| (PVP K30) | | | | |
| Glycerol | Plasticiser | 0.1 | 0.74 | 11 |
| Polyethylene | Plasticiser | 0.042 | 0.31 | 4.6 |
| Glycol | | | | |
| (PEG 1500) | | | | |
| Spearmint oil | Flavouring | 0.07 | 0.56 | 7.7 |
| flavour | agent | | | |
| Drug active | Active | 0.0001-0.1 | 0.0007-0.74 | 0.011-10.95 |
| Distilled Water | Solvent | 12.6 | 93.26 | 0.1 |

The presence of an active pharmaceutical ingredient (API) in the formulation in the described range was not found to adversely affect the properties of the resulting oral thin film. It will be appreciated that reason for the presence of a small amount of water in the final “dry” film is that some residual water will be present within the film after casting. The amount of residual water may depend on the combination and amounts of the various ingredients used in the formulation.

It will be understood that the present embodiments are provided by way of example only, and that various modifications can be made to the present embodiments without departing from the scope of the invention.

1. A composition for an oral thin film, wherein the composition comprises:
(i) polyvinyl alcohol;
(ii) glycerol; and
(iii) polyethylene glycol of an average molecular weight ranging from about 1100 to about 2900 dalton or g·mol⁻¹ or about 1,300 to about 2,000 g·mol⁻¹,
wherein the combined amount of glycerol and polyethylene glycol is about 12 to 24 wt % in the dry formulation and the glycerol and polyethylene glycol are in a weight ratio of about 1:2.1 to 1:4.

2. The composition of claim 1, wherein the composition comprises about 30 to about 50 wt % dry weight of polyvinyl alcohol or from about to about 18 wt % wet weight of polyvinyl alcohol.

3. The composition of claim 1, wherein the composition comprises about 3 to about 7 wt % dry weight of glycerol or from about 1 to about 6 wt % wet weight of glycerol.

4. The composition of claim 1, wherein the composition comprises about 3 to about 17 wt % dry weight of polyethylene glycol or from about 1.1 to about 6.3 wt % wet weight of polyethylene glycol.

5. The composition of claim 1, further comprising hydroxypropyl methylcellulose, optionally
wherein the composition comprises about 10 to about 15 wt % dry weight of hydroxypropyl methylcellulose or from about 3.1 to about 4.7 wt % wet weight of HPMC.

6. A composition for an oral thin film, wherein the composition comprises:
(i) hydroxypropyl methylcellulose;
(ii) polyvinyl pyrrolidone;
(iii) glycerol; and
(iv) polyethylene glycol.

7. (canceled)

8. A composition for an oral thin film, wherein the composition comprises:
(i) hydroxypropyl methylcellulose;
(ii) polyvinyl pyrrolidone; and
(iii) glycerol; and/or polyethylene glycol,
wherein the composition comprises about 60 to about 66 wt % hydroxypropyl methylcellulose in the dry formulation and/or about 4 to about 8 wt % polyvinyl pyrrolidone in the dry formulation, and wherein the composition does not comprise an emulsifier.

9. The composition of claim 8, wherein the hydroxypropyl methylcellulose comprises about 19 to about 24 wt % methoxy groups and about 7 to about 12 wt % hydroxypropyl groups.

10. The composition of claim 6, wherein the hydroxypropyl methylcellulose has a dynamic viscosity of about 3500 to about 5600 cPs at 20° C. in a 2% aqueous solution.

11. The composition of claim 6, wherein the composition comprises about 60 to about 66 wt % dry weight of hydroxypropyl methylcellulose, or about 4.1 to about 4.5 wt % wet weight of hydroxypropyl methylcellulose.

12. The composition of claim 6, wherein the composition comprises about 4 to about 8 wt % dry weight of polyvinyl pyrrolidone or about 0.27 to about 0.54 wt % wet weight of polyvinyl pyrrolidone.

13. The composition of claim 6, wherein the composition comprises about 4 to about 20% dry weight of glycerol or about 0.2 to about 1.4 wt % wet weight of glycerol; and/or
wherein the composition comprises about 4 to about 20% dry weight of polyethylene glycol or about 0.2 to about 1.4 wt % wet weight of polyethylene glycol.

14. The composition of claim 6, wherein the composition comprises glycerol and polyethylene glycol in a combined amount of about 12 to about 24 wt % in the dry formulation or of 0.5 to about 1.5 wt % in the wet formulation; and/or
wherein the composition comprises polyethylene glycol and glycerol in a weight ratio of about 1:1.5 to about 1:4, such as 1:2.1 to about 1:4.

15. The composition of claim 6, wherein the polyvinyl pyrrolidone has a dynamic viscosity of about 5.5 to about 8.5 cPs at 20° C. in a 10% aqueous solution.

16. The composition of claim 1, wherein the composition does not comprise a thickener.

17. The composition of claim 1, wherein the composition does not comprise an emulsifier.

18. The composition of claim 1, wherein the composition comprises a pharmaceutically acceptable solvent, optionally wherein the solvent is water, optionally wherein the composition comprises about 60 to about 95 wt % solvent of the total (wet) composition.

19. The composition of claim 1, further comprising one or more ingredients selected from the list consisting of: a salivary stimulant; a sweetener; and a flavouring agent.

20. The composition of claim 1, further comprising a pharmaceutically active ingredient, optionally wherein the pharmaceutically active ingredient is an opioid.

21.-22. (canceled)
